(19) United States
(12) Patent Application Publication
(10) Pub. No.: US 2026/0061180 A1
(43) Pub. Date: Mar. 5, 2026

(54) HEART FAILURE ASSISTANCE

(71) Applicant: SHEBA IMPACT LTD., Ramat Gan (IL)

(72) Inventor: Leonid STERNIK, Savyon (IL)

(21) Appl. No.: 19/105,860

(22) PCT Filed: Aug. 24, 2023
(86) PCT No.: PCT/IL2023/050898

§ 371 Date: Feb. 24, 2025

(30) Foreign Application Priority Data
Aug. 24, 2022 (IL) 295899

(51) Int. Cl. A61M 60/178

(52) U.S. Cl. CPC A61M 60/178

(57) ABSTRACT

The present disclosure provides a solution for assisting cardiac circulation of a subject. In one aspect of the present disclosure, the new solution replaces the common practice of implementing a cannula of left ventricular assist device (LVAD) to the left ventricle (LV) through a large opening made in the apex of the LV, which has some disadvantages such as requirement of sternotomy, making a hole in an already very weak left ventricle, requirement of cardiopulmonary bypass, etc.

The solution of the present disclosure suggests introducing the inflow cannula, namely the cannula/tube that suctions blood from the heart, through an opening made in the left atrium appendage (LAA) and guiding it through the left atrium (LA) and the mitral valve (MV) into the LV to be positioned therein. The inflow cannula is designed to allow suction of blood along various portions of its length that are intended to be positioned within the heart, namely along portions that are positioned in the LV, LA and optionally the LAA.

The blood that is suctioned by the inflow cannula flows into a mechanical circulatory support device to be pumped therefrom, via an outflow cannula and into the aorta, thereby allowing supply of oxygenated blood to the entire body. Optionally, the outflow cannula can be coupled to the descending aorta and not to the ascending aorta as commonly done in the cases of LVAD implementations.

A second aspect of the present disclosure provides a new solution for overcoming right ventricle failure by withdrawing blood from a first portion of the pulmonary artery and streaming blood to a second portion of the pulmonary artery, the second portion is downstream the first portion, namely more proximal to the lungs. The two solutions may be carried out by a similar surgical procedure, such as left thoracotomy. If the two solutions of the present disclosure are used together, they form a total artificial heart solution.

### TECHNOLOGICAL FIELD

The present disclosure is in the cardiological field.

### BACKGROUND ART

References considered to be relevant as background to the presently disclosed subject matter are listed below:
Kiamanesh, Omid, et al. “Left ventricular assist device with a left atrial inflow cannula for hypertrophic cardiomyopathy.” Case Reports 2.13 (2020): 2090-2094.
Miyagi, Chihiro, et al. “Left Atrial Circulatory Assistance in Simulated Diastolic Heart Failure Model: First in Vitro and in Vivo.” Journal of Cardiac Failure 28.5 (2022): 789-798.
Marey, Gamal M., et al. “Berlin Excor Cannulation of Left Atrial Appendage in Left Ventricular Restrictive Physiology: A Novel Bailout Strategy.” Asaio Journal 67.9 (2021): e157.

Acknowledgement of the above references herein is not to be inferred as meaning that these are in any way relevant to the patentability of the presently disclosed subject matter.

### GENERAL DESCRIPTION

The present disclosure provides a solution for assisting cardiac circulation of a subject. In one aspect of the present disclosure, the new solution replaces the common practice of implementing a cannula of left ventricular assist device (LVAD) to the left ventricle (LV) through a large opening made in the apex of the LV, which has some disadvantages such as requirement of sternotomy, making a hole in an already very weak left ventricle, requirement of cardiopulmonary bypass, etc.

The solution of the present disclosure suggests introducing the inflow cannula, namely the cannula/tube that suctions blood from the heart, through an opening made in the left atrium appendage (LAA) and guiding it through the left atrium (LA) and the mitral valve (MV) into the LV to be positioned therein. The inflow cannula is designed to allow suction of blood along various portions of its length that are intended to be positioned within the heart, namely along portions that are positioned in the LV, LA and optionally the LAA.

The blood that is suctioned by the inflow cannula flows into a mechanical circulatory support device, such as left ventricular assist device (LVAD), temporary ventricle assist device (VAD) and extracorporeal membrane oxygenation (ECMO), to be pumped therefrom, via an outflow cannula and into the aorta, thereby allowing supply of oxygenated blood to the entire body. Optionally, the outflow cannula can be coupled to the descending aorta and not to the ascending aorta as commonly done in the cases of LVAD implementations. The inflow cannula and the outflow cannula are made of materials allowing them to be maintained implanted for the desired amount of time, which in some cases can be many years.

Due to the unique solution of the present invention, in which there are no holes or manmade damages to the LV, it is relatively easy to replace an old implanted mechanical circulatory support device, such as LVAD, with a new one or with a new different mechanical circulatory support device, such as ECMO.

Therefore, a first aspect of the present disclosure provides a method for assisting cardiac circulation of a subject. The method comprising introducing a distal end of a first inflow cannula into the left ventricle (LV) of the subject through the left atrial appendage (LAA), the left atrium (LA) and the mitral valve (MV) in this order. Namely, the first inflow cannula is introduced into the heart through the LAA using standard surgical means. The first inflow cannula extends between said distal end and a proximal end thereof, which is coupled to an inlet of a first mechanical circulatory support (MCS) device, e.g. LVAD or ECMO, to allow suction of blood from at least one of the LV, the LA, or both the LV and the LA, and optionally from the LAA to the first MCS device. The suction is allowed due to perforations, cuts, or any other suitable means that are formed along different portions of the first inflow cannula.

The method further comprises fluidically coupling a distal end of a first outflow cannula with the aorta. A proximal end of said first outflow cannula is coupled with an outlet of said first MCS device to allow outflow of blood from the first MCS towards the aorta. The coupling of the proximal end of the outflow cannula with the outlet of the first MCS device can be done prior or after to the coupling of the distal end of the first outflow cannula with the aorta.

In general, it is to be noted that the different steps of the method are not limited to be performed in their order of appearance.

It is to be noted that any combination of the described embodiments with respect to any aspect of this present disclosure is applicable. In other words, any aspect of the present disclosure can be defined by any combination of the described embodiments.

In some embodiments of the method, said first MCS device is selected from any one of: a left ventricular assist device (LVAD), temporary ventricle assist device (VAD) and extracorporeal membrane oxygenation (ECMO).

In some embodiments of the method, said distal end of the first outflow cannula is coupled to the descending aorta.

In some embodiments of the method, said distal end of the first outflow cannula is coupled to the ascending aorta.

In some embodiments of the method, a distal end portion of the first inflow cannula is formed with perforations for allowing suction of blood from various spots along the distal end portion. The perforations are formed along a distal end portion that is, typically, extending along the LAA, LA, MV and LA. The perforations may be uniformly/non uniformly distributed along the cannula and may have constant or variable dimensions/size.

In some embodiments of the method, said introducing comprises maintaining said perforations blocked until the distal end portion is soaked with blood to prevent air embolism during LA/LV cannula insertion.

In some embodiments of the method, said maintaining comprises inserting an internal sleeve to an internal lumen of the first inflow cannula to cover and block said perforations, wherein extraction of the internal sleeve causes unblockage of said perforations.

In some embodiments, the method further comprising placing a pump element of the first MCS device in the left thorax. This can be typically done by a left thoracotomy or by video assisted mini-thoracotomy. By placing the pump element of the first MCS device in the left thorax, a bigger pump can be used as the left thorax provides more space for such element.

In some embodiments of the method, the first inflow cannula comprises a proximal portion and a distal portion. The proximal portion is intended to be extended between the inlet of the first MCS device and up to the entrance to the LAA, while the distal portion is intended to be extended between the entrance to the LAA and the LV. The distal end portion has a cross-sectional dimension smaller than the cross-sectional dimension of the proximal portion.

In some embodiments of the method, a distal end portion of the first inflow cannula is tapered towards the distal end, defining a tapered end portion. Namely, the internal lumen dimension of the cannula, e.g. its diameter, reduces from a certain position towards the distal end.

In some embodiments of the method, said tapered end portion extends between the LAA and the LA.

In some embodiments of the method, said tapered end portion extends between the LAA and the LV.

In some embodiments, the portion of the first inflow cannula intended to be positioned in the LA is thicker, namely has a larger flow rate capacity than the portion of the first inflow cannula intended to be positioned in the LV.

In some embodiments of the method, the method further comprising sewing said first inflow cannula to the LAA defining LAA sewn portions.

In some embodiments, the method further comprising reinforcing said LAA sewn portions with rings.

In some embodiments of the method, said sewn portions comprises tissue growth inducing material.

In some embodiments of the method, the first outflow cannula comprises portions comprising tissue growth inducing material. In particular, these portions are next to the coupling portion to the aorta.

In some embodiments of the method, said introducing comprises either performing side-biting clamp of the LAA following by suturing the first inflow cannula to the LAA or performing purse-string suturing of the first inflow cannula to the LAA. Furthermore, said fluidically coupling comprises either performing side-biting clamp of the aorta following by suturing the first outflow cannula to the aorta or performing purse-string suturing of the first outflow cannula to the aorta.

In some embodiments, the method comprising implanting the first MCS device in the body of the subject. Namely, after the surgical procedure is done, the first MCS device remains totally implanted in the body of the subject and unreachable under the skin.

Yet another aspect of the present disclosure provides a method for assisting cardiac circulation of a subject. The method comprising: blocking blood flow in a selected blocking portion of the pulmonary artery (PA), e.g. by a clamp or any other known means; fluidically coupling, namely performing a cannulation, a first end of a second inflow cannula to an upstream portion of the PA upstream said blocking portion, namely the upstream portion is more proximal to the right ventricle than the blocking portion, and fluidically coupling a second end of the second inflow cannula to an inlet of a second mechanical circulatory support (MCS) device to allow suction of blood from the pulmonary artery, and therefore blood is practically suctioned from the right ventricle (RV), to the second MCS device; fluidically coupling a first end of a second outflow cannula to a downstream portion of the PA downstream said blocking portion, namely the downstream portion is more proximal to the lungs than the blocking portion, and fluidically coupling a second end of the second outflow cannula to an outlet of said second MCS device, thereby allowing outflow of blood from the MCS towards the downstream portion of the PA.

In some embodiments of the method, said second MCS device is selected from any one of: a right ventricular assist device (RVAD), temporary ventricle assist device (VAD) and extracorporeal membrane oxygenation (ECMO).

In some embodiments, the method further comprising placing a pump element of the second MCS device in the left thorax. This can be typically done by a left thoracotomy or by video assisted mini-thoracotomy. By placing the pump element of the first MCS device in the left thorax, a bigger pump can be used as the left thorax provides more space for such element.

In some embodiments, the method comprising implanting the second MCS device in the body of the subject. Namely, after the surgical procedure is done, the second MCS device remains totally implanted in the body of the subject and unreachable under the skin.

In some embodiments, the method further comprises the method of any one of the above-described embodiments, or any combination thereof, of the method related to suction of blood from the LV.

In some embodiments of the method, the first MCS is the second MCS. Namely, there is only one MCS that serves for blood suction from the LV and from the RV simultaneously and practically serves as a total artificial heart.

Yet another aspect of the present disclosure provides a kit for use in a method for assisting cardiac circulation of a subject. The kit comprising: (i) a first inflow cannula extending between an inflow cannula proximal end, configured to be coupled to an inlet of a first mechanical circulatory support (MCS) device, and an inflow cannula distal end, configured to be inserted through the left atrial appendage and the mitral valve to be placed in the left ventricle (LV) to allow suction of blood from either the LV, the LA, or the LV and the LA to the MCS device by any suitable means, such as perforations, certain profile of slits, cuts, etc.; (ii) a first outflow cannula extending between an outflow cannula proximal end, configured to be coupled to an outlet of a MCS device, and an outflow cannula distal end configured to be coupled to or inserted into the descending aorta so as to allow outflow of blood from the first MCS device to the aorta. The method comprises (1) introducing said inflow cannula distal end into the left ventricle (LV) of the subject through the left atrial appendage (LAA), the left atrium (LA) and the mitral valve (MV), (2) fluidically coupling said outflow cannula distal end with the aorta, (3) coupling said inflow cannula proximal end to the inlet of the first MCS device, and (4) coupling said outflow cannula proximal end to the outlet of the first MCS device.

In some embodiments, the kit further comprising connectors for allowing said coupling of the inflow cannula proximal end to the inlet of the first MCS device and the outflow cannula proximal end to the outlet of the first MCS device.

In some embodiments of the kit, the connectors are adapted for connection with multiple types of MCS devices.

In some embodiments, the kit further comprising said first MCS device.

In some embodiments of the kit, said first MCS device is selected from any one of: a left ventricular assist device (LVAD), temporary ventricle assist device (VAD) and extracorporeal membrane oxygenation (ECMO).

In some embodiments of the kit, said method comprising placing a pump element of the MCS device in the left thorax, performed by a left thoracotomy.

In some embodiments of the kit, said fluidically coupling comprises fluidically coupling said outflow cannula distal end with the descending aorta.

In some embodiments of the kit, a distal end portion of said first inflow cannula is formed with perforations for allowing suction of blood from various spots along the distal end portion. The perforations are formed along a distal end portion that is, typically, extending along the LAA, LA, MV and LA. The perforations may be uniformly/non uniformly distributed along the cannula and may have constant or variable dimensions/size.

In some embodiments, the kit further comprising a sleeve element configured to be fitted within an internal lumen of the first inflow cannula to block said perforations, wherein the method comprises maintaining the sleeve element within said internal lumen until said perforations are soaked with blood and then extracting it to unblock said perforations, thereby preventing air embolism.

In some embodiments of the kit, the first inflow cannula comprises a proximal portion and a distal portion. The proximal portion is intended to be extended between the inlet of the first MCS device and up to the entrance to the LAA, while the distal portion is intended to be extended between the entrance to the LAA and the LV. The distal end portion has a cross-sectional dimension smaller than the cross-sectional dimension of the proximal portion.

In some embodiments of the kit, a distal end portion of the first inflow cannula is tapered towards the distal end, defining a tapered end portion. Namely, the internal lumen dimension of the cannula, e.g. its diameter, reduces from a certain position towards the distal end. The tapered end portion is intended to be placed along the LAA, LA, MV and LA.

In some embodiments, the portion of the first inflow cannula intended to be positioned in the LA is thicker, namely has a larger flow rate capacity than the portion of the inflow cannula intended to be positioned in the LV.

In some embodiments of the kit for use in a method, the method is any one of the above-described methods.

In some embodiments of the kit, the first inflow cannula comprises portions comprising tissue growth inducing material. In some embodiments, at least some of these portions are the coupling portions to the LAA and/or in proximity to that.

In some embodiments of the kit, the first outflow cannula comprises portions comprising tissue growth inducing material. In particular, these portions are next to the coupling portion to the aorta.

In some embodiments of the kit, the MCS is designed to be implanted in the body of the subject and to remain there during its operation.

Yet another aspect of the present disclosure provides an inflow cannula for use in suctioning blood from the left ventricular (LV) and/or the left atrium (LA). The inflow cannula extends between an inflow cannula proximal end, configured to be coupled to an inlet of a mechanical circulatory support (MCS) device, and an inflow cannula distal end, configured to be placed in the LV to allow suction of blood from the LV and/or the LA to the MCS device by any suitable means, such as perforations, certain profile of slits, cuts, etc.

In some embodiments of the inflow cannula, a distal end portion of said inflow cannula is formed with perforations for allowing suction of blood from various spots along the distal end portion. The perforations are formed along a distal end portion that is, typically, extending along the LAA, LA, MV and LA.

In some embodiments, the inflow cannula further comprising a sleeve element configured to be fitted within an internal lumen of the inflow cannula to block said perforations, wherein the method comprises maintaining the sleeve element within said internal lumen until said perforations are soaked with blood and then extracting it to unblock said perforations, thereby preventing air embolism.

In some embodiments, the inflow cannula comprises a proximal portion and a distal portion. The proximal portion is intended to be extended between the inlet of the MCS device and up to the entrance to the LAA, while the distal portion is intended to be extended between the entrance to the LAA and the LV. The distal end portion has a cross-sectional dimension smaller than the cross-sectional dimension of the proximal portion.

In some embodiments of the inflow cannula, it is being tapered towards the distal end, defining a tapered end portion. Namely, the internal lumen dimension of the cannula, e.g. its diameter, reduces from a certain position towards the distal end. The tapered end portion is intended to be placed along the LAA, LA, MV and LA.

In some embodiments of the inflow cannula, it is being used in a method for assisting cardiac circulation of a subject. The method comprises introducing said inflow cannula distal end into the left ventricle (LV) of the subject through the left atrial appendage (LAA), the left atrium (LA) and the mitral valve (MV) and coupling said inflow cannula proximal end to the inlet of the MCS device.

Yet another aspect of the present disclosure provides a kit for assisting cardiac circulation of a subject. The kit comprising: a second inflow cannula for fluidically coupling a first end thereof to an upstream portion of a pulmonary artery (PA) of the subject upstream a blocking portion of the PA in which flow of blood is blocked, namely the upstream portion is more proximal to the right ventricle than the blocking portion, and for fluidically coupling a second end thereof to a second mechanical circulatory support (MCS) device to allow suction of blood from the pulmonary artery and therefore blood is suctioned from the right ventricle (RV) to the second MCS device; a second outflow cannular for fluidically coupling a first end thereof to a downstream portion of the PA downstream said blocking portion, namely the downstream portion is more proximal to the lungs than the blocking portion, and fluidically coupling a second end of thereof to an outlet of said second MCS device, thereby allowing outflow of blood from the MCS towards the downstream portion of the PA.

In some embodiments, the kit further comprises said second MCS device.

In some embodiments of the kit, said second MCS device is selected from any one of: a right ventricular assist device (RVAD), temporary ventricle assist device (VAD) and extracorporeal membrane oxygenation (ECMO).

In some embodiments, the kit further comprises a blocking element, e.g. a clamp, for blocking the blood flow in the PA in said selected blocking portion.

In some embodiments, the kit is for use in accordance with any one of above-described embodiments, or any combination thereof, of the method related to blood pumping from and to the pulmonary artery.

In some embodiments, the kit further comprises the kit of any one of the above-described embodiments, or any combination thereof, of the kit related to blood suction from the LV through the LAA.

In some embodiments of the kit, the first MCS is the second MCS. Namely, there is only one MCS that serves for blood suction from the LV and from the RV simultaneously and practically serves as a total artificial heart.

In some embodiments, the kit further comprising connectors for allowing said coupling of the second inflow cannula to the inlet of the second MCS device and the second outflow cannula to the outlet of the first MCS device.

In some embodiments of the kit, the connectors are adapted for connection with multiple types of MCS devices.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a schematic illustration of a non-limiting example showing the elements of the kit, according to an aspect of the present, disclosure implanted in a human body according to a method of the present disclosure.

FIG. 2 is a schematic illustration of a non-limiting example exemplifying the use of the kit of an aspect of the present disclosure.

### DETAILED DESCRIPTION

The following figures are provided to exemplify embodiments and realization of the invention of the present disclosure.

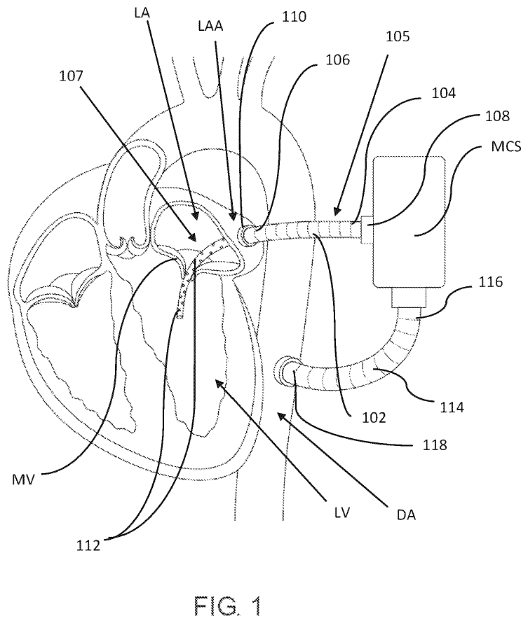

Reference is being made to FIG. 1, which is a schematic illustration of a non-limiting example showing the elements of the kit of the present disclosure implanted in a human body. FIG. 1 shows a portion of the heart of the subject including the left ventricle LV, the left atrium LV and the left atrium appendage LAA and further shows a portion of the descending aorta DA. The mitral MV is also demonstrated between the LA and the LV. An inflow cannula 102 is coupled at its cannula proximal end 104 to a mechanical circulatory support device MCS and is introduced via the LAA, the LA and the MV such that its distal end 106 is positioned in the LV. Namely, there a first portion 105 of the inflow cannula 102 is spanned over the LV, MV, LA and the LAA and a second portion 107 is located outside the heart of the subject that ends in a coupling mechanism 108 with the MCS. The coupling mechanism 108 is configured to allow coupling of the inflow cannula 102 to any type of MCS such as left ventricular assist device (LVAD), temporary ventricle assist device (VAD) and extracorporeal membrane oxygenation (ECMO). The inflow cannula 102 is introduced to the heart via the LAA and sutured thereto by standard surgical methods. This can be carried out by purse-string suturing or by side-biting clamp. The suture portion of the LAA to the inflow cannula 102 can be reinforced by a reinforcing element 110 that typically induces tissue growth to reinforce the suture portion. The second portion 107, namely the portion of the inflow cannula 102 that extends between the suture portion and the distal end 106, has a smaller dimension, e.g. a smaller diameter, than that of the first portion 105, which extends between the suture portion and the proximal end 104. The second portion 107 can have a constant dimension or gradually reducing dimension that ends in a tapered edge at the distal end 106. The second portion 107 is formed with perforations 112, allowing suction of blood from various sections of the second portion 107, i.e. suction of blood from the LV, the LA and/or the LAA. At the introduction step of the inflow cannula, the perforations can be blocked, e.g. by an internal removable sleeve, for avoiding suction of air that can lead to air embolism. Once the perforated portion of the inflow cannula is soaked and surrounded with blood, the internal sleeve is removed, exposing the perforations and allowing suction of blood therefrom. The suctioned blood is transferred to the MCS and is pumped to the descending aorta DA through an outflow cannula 114 that is coupled to an outlet of the MCS at a proximal end thereof 116 and to the DA at distal end thereof 118. The coupling of the outflow cannula 114 to the DA can be carried out by standard surgical methods, such as purse-string suturing or by side-biting clamp.

The present invention is provided in view of some disadvantages of known method of LVAD implementations, such as:
Conducting cardiopulmonary bypass.

Conducting sternotomy. In some cases, an LVAD implanted in patients which underwent open heart surgery (in most cases sternotomy). In other cases, a heart transplantation can be done after LVAD. In all these cases redo sternotomy is increasing operative risk.

Piercing a hole in the left ventricle and sewing the device to the left ventricle, that is already very weak and poorly contracting.

Thus, the present invention provides a solution that:
Can be implanted easily without cardiopulmonary bypass
Implanted through the left thoracotomy and can be done without redo sternotomy in patients with cardiac surgery in the past or heart transplant after LVAD.
Requires no holes, suturing or other manipulation of the left ventricle.
Enables inflow and outflow connection of the LVAD to ECMO.

Therefore, in a non-limiting example, the present invention provides LVAD that it is placed through the left thoracotomy or video assisted minithoracothomy (VATS). Inflow cannula is placed through the purse string or other similar device to the left atrium through the left atrial appendage. It's distal and small and narrow part is placed into the left ventricle through the mitral valve under ECHO TEE or X-ray control inter alia to prevent blood clot formation in the ventricle. The outflow cannula is inserted into the descending aorta through the purse string or other similar device. Inflow and outflow cannulas can be also connected to the left atrium and aorta with grafts which can be sewn or connected by other means to left atrium or aorta. If an inflow graft is sewn to the left atrial appendage (left atrium) the graft may be reinforced with rings, for example and importantly a small cannula, 14 Fr for example, may be inserted through the mitral valve into the left ventricle to suck blood, decompress the left ventricle and prevent blood clot formation. The inflow and outflow graft have one end to be sewn to left atrium (appendage) or aorta and another end with a special connector to be connected to LVAD or ECMO.

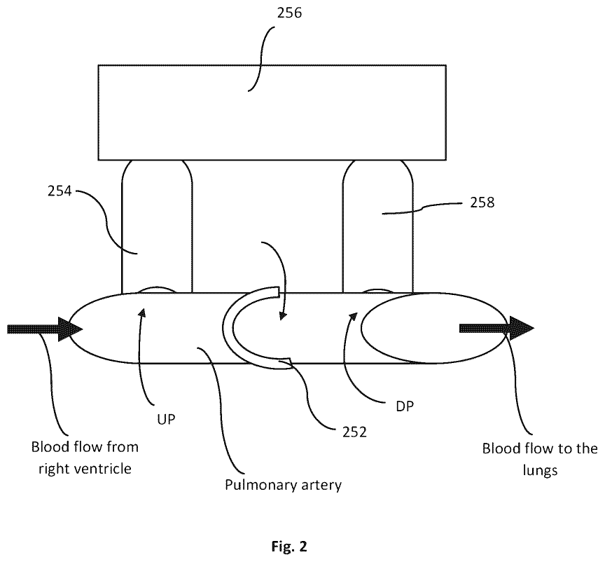

Reference is now being made to FIG. 2, which is a schematic exemplifying the use of the kit for assisting the functionality of the right ventricle of a subject. The figure shows a part of the pulmonary artery (PA) of a subject. It is to be noted that the figure is schematic for the explanation of the technique and the shape of the PA does not necessarily resemble the real shape of PA, though the skilled in the art should understand from this schematic illustration how to realize the invention. The part of the PA has a blocking portion BP that is blocked by a blocking element 252, such as a clamp. No blood flows (or very little flows) naturally from one side of the blocking portion BP to the other side thereof, namely from the right ventricle side to the lungs side. An inflow cannula 254 is coupled with a first end thereof to an upstream portion UP of the PA that is upstream the blocking portion BP. A second end of the inflow cannula is coupled to an inlet of a mechanical circulatory support (MCS) device 256, thereby the inflow cannula 254 allowing suction of blood from the upstream portion UP to the MCS device 256 that results in a suction of blood from the right ventricle (RV) of the subject. An outflow cannula 258 is coupled with a first end thereof to a downstream portion DP and with a second end thereof to an outlet of the MCS device 256, thereby allowing blood flow from the MCS device 256 to the downstream portion 256 and then to the lungs. By forming this setup, the MCS device 256 actually pumps blood from the RV to the lungs. The MCS device may be selected from any one of: a right ventricular assist device (RVAD), temporary ventricle assist device (VAD) and extracorporeal membrane oxygenation (ECMO). The MCS may be totally implanted in the body of the subject or to be held outside the body.

This setup can be performed by a left thoracotomy, which is relatively minimalistic procedure with respect to other known alternatives, such as sternotomy. It is to be noted that the coupling of the cannulas to the PA can be made by a cannulation technique or any other known in the art technique.

The setup provided by FIG. 2 can be carried out together with the setup provided by FIG. 1, when the MCS devices can be separated or can be combined to a single MCS device that can provide pumping capabilities to both the LV and the RV simultaneously.

1-37. (canceled)

38. A method for assisting cardiac circulation of a subject, comprising:
introducing a distal end of a first inflow cannula into the left ventricle (LV) of the subject through the left atrial appendage (LAA), the left atrium (LA) and the mitral valve (MV), the first inflow cannula extending between said distal end and a proximal end being coupled to an inlet of a mechanical circulatory support (MCS) device to allow suction of blood from the LV and/or the LA, to the MCS device;
fluidically coupling a distal end of a first outflow cannula with the aorta, a proximal end of said first outflow cannula is coupled with an outlet of said MCS device to allow outflow of blood from the MCS towards the aorta.

39. The method of claim 38, wherein said first or second MCS device is selected from any one of: a left ventricular assist device (LVAD), temporary ventricle assist device (VAD) and extracorporeal membrane oxygenation (ECMO).

40. The method of claim 38, wherein said distal end of the first outflow cannula is coupled to the descending aorta.

41. The method of claim 38, wherein a distal end portion of the first inflow cannula is formed with perforations;
wherein said introducing comprises maintaining said perforations blocked until the distal end portion is soaked with blood;
wherein said maintaining comprises inserting an internal sleeve to an internal lumen of the first inflow cannula to cover and block said perforations, wherein extraction of the internal sleeve causes unblockage of said perforations.

42. The method of claim 38, comprising placing a pump element of the first MCS device in the left thorax.

43. The method of claim 38, wherein a distal end portion of the first inflow cannula is tapered towards the distal end, defining a tapered end portion;
wherein said tapered end portion extends between the LAA and the LA.

44. The method of claim 38, wherein the method further comprising sewing said first inflow cannula to the LAA defining LAA sewn portions.

45. The method of claim 44, further comprising reinforcing said LAA sewn portions with rings;
wherein said sewn portions comprise tissue growth inducing material.

46. The method of claim 38, wherein said introducing comprises either performing side-biting clamp of the LAA following by suturing the first inflow cannula to the LAA or performing purse-string suturing of the first inflow cannula to the LAA; and
wherein said fluidically coupling comprises either performing side-biting clamp of the aorta following by suturing the first outflow cannula to the aorta or performing purse-string suturing of the first outflow cannula to the aorta.

47. The method of claim 38, comprising:
blocking blood flow in a selected blocking portion of the pulmonary artery (PA);
fluidically coupling a first end of a second inflow cannula to an upstream portion of the PA upstream said blocking portion and fluidically coupling a second end of the second inflow cannula to an inlet of a second mechanical circulatory support (MCS) device to allow suction of blood from the pulmonary artery to the second MCS device, resulting in a suction of blood from the right ventricle to the MCS device;
fluidically coupling a first end of a second outflow cannula to a downstream portion of the PA downstream said blocking portion and fluidically coupling a second end of the second outflow cannula to an outlet of said second MCS device, thereby allowing outflow of blood from the MCS towards the downstream portion of the PA.

48. The method of claim 47, wherein said second MCS device is selected from any one of: a left ventricular assist device (LVAD), temporary ventricle assist device (VAD) and extracorporeal membrane oxygenation (ECMO).

49. The method of claim 48, wherein the first MCS is the second MCS.

50. A kit for assisting cardiac circulation of a subject, the kit comprising:
a first inflow cannula extending between an inflow cannula proximal end, configured to be coupled to an inlet of a first mechanical circulatory support (MCS) device, and an inflow cannula distal end, configured to be inserted through the left atrial appendage and the mitral valve to be placed in the left ventricle (LV) to allow suction of blood from the LV and/or the LA to the first MCS device;
a first outflow cannula extending between an outflow cannula proximal end, configured to be coupled to an outlet of the first MCS device, and an outflow cannula distal end configured to be coupled to or inserted into the descending aorta so as to allow outflow of blood from the first MCS device to the aorta.

51. The kit of claim 50, wherein the kit is for use in a method for assisting cardiac circulation of the subject, wherein said method comprises:
introducing said first inflow cannula distal end into the left ventricle (LV) of the subject through the left atrial appendage (LAA), the left atrium (LA) and the mitral valve (MV),
fluidically coupling said first outflow cannula distal end with the aorta,
coupling said first inflow cannula proximal end to the inlet of the first MCS device, and
coupling said first outflow cannula proximal end to the outlet of the first MCS device;
wherein the method further comprises placing a pump element of the first MCS device in the left thorax.

52. The kit of claim 50, comprising connectors for allowing said coupling of the first inflow cannula proximal end to the inlet of the first MCS device and the outflow cannula proximal end to the outlet of the first MCS device.

53. The kit of claim 50, comprising said first MCS device;
wherein said first MCS device is selected from any one of: a left ventricular assist device (LVAD), temporary ventricle assist device (VAD) and extracorporeal membrane oxygenation (ECMO).

54. The kit of claim 50, wherein a distal end portion of said first inflow cannula is formed with perforations;
the kit further comprises a sleeve element configured to be fitted within an internal lumen of the first inflow cannula to block said perforations;
wherein a distal end portion of the first inflow cannula is tapered towards the distal end, defining a tapered end portion.

55. The kit of claim 50, comprising:
a second inflow cannula for fluidically coupling a first end thereof to an upstream portion of a pulmonary artery (PA) of the subject upstream a blocking portion of the PA in which flow of blood is blocked and for fluidically coupling a second end thereof to a second mechanical circulatory support (MCS) device to allow suction of blood from the pulmonary artery to the second MCS device, resulting in a suction from the right ventricle to the second MCS device;
a second outflow cannular for fluidically coupling a first end thereof to a downstream portion of the PA downstream said blocking portion and fluidically coupling a second end of thereof to an outlet of said second MCS device, thereby allowing outflow of blood from the MCS towards the downstream portion of the PA.

56. The kit of claim 55, comprising said second MCS device;
wherein said second MCS device is selected from any one of: a left ventricular assist device (LVAD), temporary ventricle assist device (VAD) and extracorporeal membrane oxygenation (ECMO).

57. The kit of claim 55, comprising a blocking element for blocking the blood flow in the PA in said selected blocking portion.
